(19) United States
(12) Patent Application Publication
(10) Pub. No.: US 2010/0310680 A1
(43) Pub. Date: Dec. 9, 2010

(54) COMPOSITION AND METHOD FOR TREATING ACNE

(76) Inventors: Theresa Chen, Hopewell, NJ (US); Geraldine Watson, Redondo Beach, CA (US); I-Ting Wu, Gardena, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/481,256

(22) Filed: Jun. 9, 2009

(51) Int. Cl. A61K 31/14; A61K 31/53; A61K 31/505; A61K 31/27; A61K 31/155; A61P 17/10; A61K 31/60; A61K 31/203; A61K 31/327; A61K 33/04; A61K 31/20; A61K 31/4025; A61K 31/192; A61K 31/7048; A61K 31/63; A61K 31/145

(52) U.S. Cl. 424/705; 514/244; 514/256; 514/479; 514/634; 514/643; 514/159; 514/559; 514/714; 514/574; 514/422; 514/569; 514/29; 514/155; 514/646

(57) ABSTRACT

This invention relates to a novel composition and method useful for the treatment of acne, and more particularly, to a composition and method for reducing an appearance of post acne marks and scars without skin irritation in a short amount of time.

### FIELD OF THE INVENTION

This invention relates to a novel composition and method useful for the treatment of skin, and more particularly, to a composition and method for reducing an appearance of post acne marks and scars without skin irritation.

### BACKGROUND OF THE INVENTION

Acne is a skin disorder that is often accompanied by pimples that sometimes cause acne sufferers embarrassment. Consumers have utilized topical anti-acne agents, such as salicylic acid and benzoyl peroxide to treat acne for many years. Topical anti-acne agents typically function by exfoliating skin and/or killing bacteria on the surface of the skin. Although such anti-acne agents are often effective at treating acne pimples, they tend to take a long time, typically days or weeks, to abate acne symptoms. They are also often ineffective at treating pre-emergent pimples.

Pigment abnormalities such as post-inflammatory hyperpigmentation (PIH) and scars are often caused by acne, pseudofolliculitis barbae (PFB), and other follicular diseases. They are more common in darker skin types, but all skin types can suffer from these post-inflammatory marks. The presence of PIH and dark marks results in skin that does not appear smooth, clear or even.

It is advantageous, therefore, to provide means for fading acne scars and provide a fast resolution in treating and preventing acne lesions. It is also advantageous to provide a composition and method for treating acne in an expeditious manner without irritating the skin.

Existing anti-acne treatments and preparations do not treat post acne marks and scars. Additionally, existing anti-acne treatments can be very irritating or harsh when used on compromised skin.

U.S. Patent Appln. Publication No. 2006/0008538A1, filed on Jun. 20, 2005, which is incorporated herein by reference, discloses compositions comprising an anti-acne agent, an anti-microbial agent and a lactate. Applicants have now discovered that compositions also containing an alpha-hydroxy acid (AHA) can reduce acne scars without irritation in limited amount of time.

Glycolic acid and other AHA's are most often used as a chemical peel administered by a dermatologist in concentrations of 20 to 80% or in at-home kits in lower concentrations of 10%. Therefore, there exists a need for a novel composition and method for treating acne and fading acne scars in a short amount of time.

### SUMMARY OF THE INVENTION

The present invention features a composition including an alpha-hydroxy acid, an anti-acne agent, an antimicrobial agent and a lactate that is capable of reducing the appearance of acne within about one week. The present invention also features a method of reducing the appearance of acne scars and marks on the skin by applying a composition to an area of skin in need of such treatment.

Other features and advantages of the present invention will be apparent from the detailed description of the invention and from the claims.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Whenever used, any percentage is weight by weight (w/w) unless otherwise indicated.

As used herein, “topically applying” means directly laying on or spreading on outer skin, e.g., by use of the hands or an applicator such as a wipe, puff, roller, or spray.

As used herein, “cosmetically-acceptable” means that the compound(s) or composition(s) which the term describes are suitable for use in contact with tissues (e.g., the skin) without undue toxicity, incompatibility, instability, irritation, allergic response, and the like. This term is not intended to limit the compound/composition it describes for use solely as a cosmetic (e.g., the ingredient/product may be used as a pharmaceutical).

As used herein, “safe and effective amount” means an amount of compound(s) or composition(s) sufficient to treat acne, but low enough to avoid serious side effects.

What is meant by “promoting” is promoting, advertising, or marketing. Examples of promoting include, but are not limited to, written, visual, or verbal statements made on the product containing the composition or in stores, magazines, newspaper, radio, television, internet, and the like.

The compositions of the present invention are useful for treating follicular diseases, such as acne, rosacea, hyperlipidemia, seborrhea, sebacious hyperplasia, follicular rash, demodex folliculorum follicular infections such as folliculitis, staphylococcal impetigoacne necrotica, and psudofolliculitis barbe, follicular ketarosis, keratosis pilaris, phrynoderma, ichthyosis follicularis, alopecia, follicular dysplasia, hirsutism, oily skin, and hypertrichosis. As used herein, the term “treating” or “treatment” means the treatment (e.g., alleviation or elimination of symptoms and/or cure) and/or prevention or inhibition of the condition (e.g., a skin condition).

The compositions of the present invention are also useful for evening skin tone (such as lightening dark areas of skin) in need of such treatment, smoothing the skin (such as reducing texture on the skin), reducing the production of sebum, and reducing the appearance of oil, shine, and/or pores on skin in need of such treatment. Examples of such skin in need of such treatment include, but are not limited to, skin having excessive pigmentation (such as freckles, post-inflammatory hyperpigmentation (PIH), or pigmented scars), rough skin, oily skin, or skin having large, visible pores.

In one embodiment, the composition is for the treatment of acne, including but not limited to the treatment or prevention of acne blemishes, acne pimples, pre-emergent pimples, blackheads, and/or whiteheads. What is meant by a “pre-emergent pimple” is an inflamed follicle that is not visually apparent on the surface of the skin with the naked eye (e.g., as a lesion).

In one embodiment, the appearance of acne (e.g., the size and/or elevation/swelling of the acne lesion and/or blackhead) is reduced within about eight hours, such as within about four hours of treatment with the present composition. In another embodiment, the size of an acne lesion treated with the present composition is reduced by about 6% within about 4 hours of treatment with the present composition.

The compositions of the present invention include an alpha-hydroxy acid. Examples of alpha-hydroxy acids include, but are not limited to: glycolic acid, lactic acid, malic acid, citric acid, tartaric acid, and combinations thereof.

In one embodiment, the amount of alpha-hydroxy acid in the compositions is from about 0.5% to about 5.0%, such as from about 1.5% to about 4.0%, such as from about 2.5% to about 3.0% by weight, based on the total weight of the composition.

The compositions include an anti-acne agent. What is meant by an anti-acne agent is a compound that has been approved by the U.S. Food and Drug Administration for the topical treatment of acne. Examples of anti-acne agents include, but are not limited to, salicylic acid, benzoyl peroxide, sulphur, retinoic acid, candida bombicola/glucose/methyl rapeseedate ferment, peat water, resorcinol, silt, peat, permethin, azelaic acid, clindamycin, adapalene, erythromycin, sodium sulfacetamide, dapsone and combinations thereof. In one embodiment, the amount of anti-acne agent in the composition is from about 0.01% to about 10%, for example from about 0.1% to about 5%, or from about 0.5% to about 2% by weight, based on the total weight of the composition.

The compositions of the present invention include an antimicrobial agent. What is meant by an antimicrobial agent is a compound that kills microorganisms or prevents or inhibits their growth or reproduction. Examples of antimicrobial agents include, but are not limited to: ethanol, propanol, betains, benzalkonium chloride, benzethonium chloride, lauric arginayte, sugarquat, methyl benzethonium chloride, cetypyridiunium chloride, 2,4,4′,-trichloro-2-hydroxy diphenyl ether (Triclosan), parachlorometa xylenol (PCMX), Iodopropynyl butylcarbamate, diazolidinyl urea, chlorhexidene digluconate, chlorhexidene acetate, chlorhexidene isethionate, chlorhexidene hydrochloride, hexetidine, Quaternium 15, triclocarbon, polyhexamethylene biguanide, cetylpyridium chloride, imidazolidinyl urea, diazolidinyl urea, 3-iodo-2-propynyl-N-butylcarbamate, 2-methyl-4-isothiazolin-3-one, dimethyl dimethyl hydantoin, (5-chloro-2-(2,4-dichlorophenoxy)phenol, monolaurin glyceryl laurate, camellia sinensis, candida bombicola/glucose/methyl rapeseedate ferment, hydrogen peroxide, phenol, poloxamer 188, PVP-iodine, thiourea, natural antimicrobial agents, such as cinnamon oil, cinnamaldehyde, lemongrass oil, clove oil, saw palmetto extract, thyme oil white, thyme oil red, thymol, tea tree oil, pinus pinaster bark extract, rosemary leaf extract, grape seed extract, and betel oil, silver containing compounds, such as silver nitrate, silver lactate, silver citrate, and silver zeolite, antimicrobial fatty acid ester of a polyhydric alcohol, a fatty ether of a polyhydric alcohol and alkoxylated derivatives thereof, and combinations thereof.

In one embodiment, the amount of antimicrobial agent in the compositions is from about 0.001% to about 10%, such as from about 0.01% to about 5%, such as from about 0.05% to about 2% by weight, based on the total weight of the composition.

In one embodiment the antimicrobial agent is an anti-fungal agent such as an azole. Examples include, but are not limited to, miconazole, ketoconazole, econazole, itraconazole, sertaconazole, fluconazole, voriconazole, clioquinol, bifoconazole, terconazole, butoconazole, tioconazole, oxiconazole, sulconazole, saperconazole, clotrimazole, undecylenic acid, haloprogin, butenafine, tolnaftate, nystatin, ciclopirox olamine, terbinafine, amorolfine, naftifine, elubiol, griseofulvin, their cosmetically acceptable salts, and combinations thereof.

In one embodiment the antimicrobial agent is an antibiotic or an antiseptic. Examples include, but are not limited to, mupirocin, neomycin sulfate bacitracin, polymyxin B, 1-ofloxacin, tetracyclines such as chlortetracycline hydrochloride, oxytetracycline-10 hydrochloride and tetrachcycline hydrochloride, clindamycin phosphate, gentamicin sulfate, metronidazole, hexylresorcinol, methylbenzethonium chloride, phenol, quaternary ammonium compounds, tea tree oil, and combinations thereof.

The composition of the present invention further includes a lactate. Examples of lactates include, but are not limited to, C2-C35 lactates such as C2-C22 lactates such as cetyl lactate and C12-C15 lactates. The amount of lactates in the composition of the present invention may vary from about 0.1% to about 50%, for example from about 0.5% to about 20%, or from about 1% to about 10% by weight, based on the total weight of the composition.

In one embodiment, the compositions of the present invention include an antipsoriatic agent. Examples of antipsoriatic agents include, but are not limited to, corticosteroids (e.g., betamethasone dipropionate, betamethasone valerate, clobetasol propionate, diflorasone diacetate, halobetasol propionate, triamcinonide, dexamethasone, fluocinonide, fluocinolone acetonide, halcinonide, triamcinolone acetate, hydrocortisone, hydrocortisone venerate, hydrocortisone butyrate, aclometasone dipropionate, flurandrenolide, mometasone furoate, and methylprednisolone acetate), methotrexate, cyclosporine, calcipotriene, anthraline, shale oil, elubiol, ketoconazole, coal tar, salicylic acid, zinc pyrithione, selenium sulfide, hydrocortisone, sulfur, menthol, and pramoxine hydrochloride, and combinations thereof.

In one embodiment, the compositions of the present invention include an anti-viral agent. Examples of anti-viral agents include, but are not limited to, imiquimod, podofilox, podophyllin, interferon alpha, acyclovir, famcyclovir, valcyclovir, reticulos and cidofovir.

In one embodiment, the compositions of the present invention include an anti-inflammatory agent. Examples of anti-inflammatory agents, include, but are not limited to, non-steroidal and steroidal anti-inflammatory agents such as corticosteroids such as hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, and triamcinolone, and combinations thereof. Examples of non-steroidal anti-inflammatory agents include but not limited to COX inhibitors, LOX inhibitors, and p38 kinase inhibitors, immunosuppressant agents such as cyclosporin, and cytokine synthesis inhibitors. Other natural anti-inflammatories include, but are not limited to, extracts of feverfew, soy, or oats, beta-glucan, and totarol.

Other active agents that may be included in the composition include, but are not limited to, wound healing enhancing agents such as calcium alginate, collagen, recombinant human platelet-derived growth factor (PDGF) and other growth factors, ketanserin, iloprost, prostaglandin E.sub.1 and hyaluronic acid; scar reducing agents such as mannose-6-phosphate; analgesic agents; debriding agents such as papain, and enzymatic debriding agents; and anesthetics such as lidocaine and benzocaine. In one embodiment, the composition comprises one or more of menthol, camphor, an antihistamine, or a local anesthetic such as tetracaine, lidocaine, prilocalne, benzocaine, bupivacaine, mepivacaine, dibucaine, etidocaine, butacaine, cyclomethycaine, hexylcaine, proparacaine, and lopivacaine, capsaicin, or oatmeal.

In one embodiment, the amount of anti-inflammatory agent, anti-viral agent, antipsoroiatic agent and/or other active agent in the compositions is from about 0.001% to about 10%, such as from about 0.01% to about 5% such as from about 0.05% to about 2% by weight, based on the total weight of the composition.

In one embodiment, the composition of the present invention further comprises a phospholipid. Examples of phospholipids include, but are not limited to synthetic phospholipids and natural phospholipids such as phospholipids composed of diester and triester phosphatides such as cocamidopropyl PG-dimonium chloride (Colalipid® C, Colonial Chemical, Inc., South Pittsburgh, Tenn., USA), stearamidopropyl PG-dimonium chloride (Colalipid® ST), sunflower amidopropyl phosphate PG-dimonium chloride (Colalipid® SUN), sodium olive amidopropyl PG-dimonium chloride phosphate (Colalipid® OL), sodium grapeseed amindopropyl PG-dimonium chloride phosphate (Colalipid® GS), linoleamidopropyl PG-dimonium chloride phosphate (Colalipid® SAFEL), PEG-8 dimethicone sunflower amidopropyl PG-dimonium complex (Colalipid® SIL), ricinoleamidopropyl PG-dimonium chloride phosphate (Colalipid® RC), sodium coco PG-dimonium chloride phosphate (Arlasilk® phospholipids CDM, Uniqema, ICI Group of Companies, Wilton, UK), cocamidopropyl PG-dimonium chloride (Arlasilk® phospholipids PTC), stearamidipropyl PG-dimonium chloride phosphate (Arlasilk® phospholipids SV), linoleamidopropyl PG-dimonium chloride phosphate (Arlasilk® phospholipids EFA), linoleamidopropyl PG-dimonium chloride phosphate dimethicone (Arlasilk® phospholipids PLN), myristamidopropyl PG-dimonium chloride phosphate (Arlasilk® phospholipids PTM), and sodium borageamidopropyl PG-dimonium chloride phosphate (Arlasilk® phospholipids GLA), and combinations thereof.

In one embodiment, the composition includes a sebum miscible agent. What is meant by a sebum miscible agent is an agent that is miscible with sebum according to the following assay. Artificial sebum is prepared as set forth on page 79 (Table 5.4) of a book chapter entitled “The Influence of Skin Surface Lipids on Topical Formulations” by Obsorne and Hatzenbuhler (in “Topical Drug Delivery Formulations”, edited by D. Osborne and A. Amann, Marcel Dekker, Inc., New York, 1990, pages 69-85). At room temperature this sebum is a white waxy substance. 50 .mu.l of the sebum is deposited into a 200 .mu.l clear vial using a precision micropipette. 100 .mu.l of the test agent is then added to the vial. The vial is warmed at 32° C. and visually inspected at the baseline and at eight hours. If the agent is miscible with the sebum, the sebum will become transparent.

The following are non-limiting examples of sebum miscible agents: aromatic alcohols such as phenyl alcohols with chemical structures of C₆H₅—R(OH) where R is an aliphatic radical, such as benzyl alcohol and phenethyl alcohol; aromatic glycol ethers such as ethylene glycol phenyl ether; propylene or butylene oxide-based glycol ethers such as propylene glycol methyl ether and those disclosed in U.S. Pat. No. 5,133,967; fatty acids, polyunsaturated fatty acids such as linoleic acid, linolenic acid, stearidonic acid, plant, fruit, or marine derived extracts rich in essential fatty acid or polyunsaturated fatty acids such as but not limited to vaccinium myrtillus (bilberry) seed oil, vaccinium macrocarpon (cranberry) seed oil, vaccinium vitis-idaea (lingonberry) seed oil, rubus idaeus (raspberry) seed oil, rubus chamaemorus (cloudberry) seed oil, ribes nigrum (black currant) seed oil, hippophae rhamnoides (sea buckthorn) seed oil, echium plantagineum (echium) seed oil, hordeum vulgare (barley) seed oil, betula alba bud extract, saw palmetto extract, borage oil, evening primrose oil, witch hazel extract and soy oil; cetyl ocenate; isostearyl benzoate; pentaerythiol teraoctenate; isostearyl benzoate; methyl gluceth; tocopherol acetate; benzalkonium chloride; and benzethonium chloride, and combinations thereof.

The compositions of the present invention may further include an alcohol. Examples of suitable alcohols include, but are not limited to, ethyl alcohol. In one embodiment, the composition includes less than 40%, such as from about 0.01% to about 40%, for example from about 0.1% to about 30%, or from about 1% to about 20% by weight, of alcohol based on the total weight of the composition.

In one embodiment, the composition includes a nonionic surfactant. Examples of nonionic surfactants are disclosed on pages 2955-2976 of the International Cosmetic Ingredient Dictionary and Handbook, eds. Wenninger and McEwen, (The Cosmetic, Toiletry, and Fragrance Assoc., Washington, D.C., 9.sup.th Edition, 2002) (hereinafter “CTFA Handbook”).

In one embodiment, the composition includes a skin lightening agent. Examples of skin lightening agents include, but are not limited to, retinoids such as retinol, extracts of soy or licorice, and tyrosine inhibitors such as hydroquinone, ascorbyl glucoside, kojic acid, calcium D-pantetheine-S-sulfonate, arbutin, magnesium ascorbyl phosphate, pantothiol, dihydrolipoic acid, and arlatone.

In one embodiment, the compositions of the present invention include a hair growth agent. What is meant by a hair growth agent is a compound that induces hair growth. Examples of hair growth agents include, but are not limited to, minoxadil, spironolactone, cyproterone acetate, azelaic acid, buserelin, bicalutamide, cromakalim, cyclosporin, aminoglutethimide, cyproterone acetate, diazoxide, phenyloin, estradiols, flutamide, prezatide copper, inocoterone, leuprolide acetate, ketoconazole, pinacidil, progesterone, finasteride, retinoic acid, turosteride, vitamins and minerals such as vitamin E, niacin (vitamin B3), pantothenic acid (vitamin B5), pyridoxine (vitamin B6), vitamin B12, vitamin C, vitamin K, biotin, inositol, zinc, copper, cysteine, methionine, coenzyme Q10, essential fatty acids such as flaxseed oil, primrose oil, and fish oil, herbal extracts such as ginko biloba, and combinations thereof.

In one embodiment, the compositions of the present invention include a hair retardation agent. What is meant by a hair retardation agent is a compound that reduces the appearance and/or growth of hair. Examples of hair retardation agents include, but are not limited to eflornithine hydrochloride, soy extracts, antiandrogenic sterols from serenoa (Serenoa repens) and/or from Cucurbita seeds (Cucurbita pepo), dipeptide N-(Carboxymethyl)phenylalanyl-.beta.-alanine compounds, 3-deazaneplanocin, inhibitors of nitric oxide synthetase such as NG-methyl-L-arginine, inhibitors of glutamine metabolism such as 6-diazo-5-oxonorleucine (I), and combinations thereof.

In one embodiment, the composition of the present invention has a pH greater than about 2 and less than about 10 such as less than about 7, such as less than about 4.5.

The topical compositions useful in the present invention comprise formulations suitable for topical application to skin. Accordingly, the composition may further include a cosmetically-acceptable topical carrier. The cosmetically-acceptable topical carrier may comprise about 50% to about 99%, by weight, of the composition (e.g., from about 80% to about 95%, by weight, of the composition).

The compositions may be made into a wide variety of product types that include but are not limited to solid and liquid compositions such as lotions, creams, gels, sticks, sprays, shaving creams, ointments, cleansing liquid washes and solid bars, pastes, powders, mousses, masks, peels, make-ups, and wipes. These product types may comprise several types of cosmetically acceptable topical carriers including, but not limited to solutions, emulsions (e.g., microemulsions and nanoemulsions), gels, solids and liposomes. The compositions may be used in conjunction with other devices such as skin abrading, skin messaging, electro-stimulation devices, light-therapy devices, ultrasound devices, radio frequency devices, thermal/cooling devices, and micro-penetration devices. The following are non-limitative examples of such carriers. Other carriers can be formulated by those of ordinary skill in the art.

The topical compositions useful in the present invention can be formulated as solutions. Solutions typically include an aqueous solvent (e.g., from about 50% to about 99% or from about 90% to about 95% of a cosmetically acceptable aqueous solvent).

Topical compositions useful in the subject invention may be formulated as a solution comprising an emollient. Such compositions preferably contain from about 2% to about 50% of an emollient(s). As used herein, “emollients” refer to materials used for the prevention or relief of dryness, as well as for the protection of the skin. A wide variety of suitable emollients are known and may be used herein. See CTFA Handbook pp. 1656-61, 1626, and 1654-55.

A lotion can be made from such a solution. Lotions typically comprise from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically comprises from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may comprise a simple base of animal or vegetable oils or semi-solid hydrocarbons. An ointment may comprise from about 2% to about 10% of an emollient(s) plus from about 0.1% to about 2% of a thickening agent(s). A more complete disclosure of thickening agents or viscosity increasing agents useful herein can be found in the CTFA Handbook pp. 1693-1697.

The topical compositions useful in the present invention may be formulated as emulsions. If the carrier is an emulsion, from about 1% to about 10% (e.g., from about 2% to about 5%) of the carrier comprises an emulsifier(s). Emulsifiers may be nonionic, anionic or cationic. Suitable emulsifiers are disclosed in, for example, CTFA Handbook, pp. 1673-1686.

Lotions and creams can be formulated as emulsions. Typically such lotions comprise from 0.5% to about 5% of an emulsifier(s). Such creams would typically comprise from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the cosmetic art and are useful in the subject invention. Multiphase emulsion compositions, such as the water-in-oil-in-water type are also useful in the subject invention. In general, such single or multiphase emulsions contain water, emollients, and emulsifiers as essential ingredients.

The topical compositions of this invention can also be formulated as a gel (e.g., an aqueous gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprise between about 0.1% and 5%, by weight, of such gelling agents.

In one embodiment, the composition is anhydrous. In one embodiment, such anhydrous composition is exothermic upon application.

The topical compositions of the present invention can also be formulated into a solid formulation (e.g., a wax-based stick, soap bar composition, powder, a wipe containing powder, or a dressing).

The topical compositions useful in the subject invention may contain, in addition to the aforementioned components, a wide variety of additional oil-soluble materials and/or water-soluble materials conventionally used in compositions for use on the skin at their art-established levels.

The topical compositions may be applied as needed and/or as part of a regular regimen ranging from application once a week up to one or more times a day (e.g., twice a day). The amount used will vary with the age and physical condition of the end user, the duration of the treatment, the specific compound, product, or composition employed, the particular cosmetically-acceptable carrier utilized, and like factors.

The following examples serve to illustrate the compositions and methods of this invention. However, they are not presented in order to limit the scope of the invention in any way.

### Example 1

An in vivo clinical study was performed using the inventive composition shown in Table 1 on 25 human volunteers. The study was double blind and randomized. The subjects suffered from acne. 3-5 acute acne lesions on the face of each volunteer were evaluated both by a clinician and the subject at baseline. The subject then applied the inventive composition one to two times per day on the lesions. The treatment site was evaluated after 4, 8 and 24 hours. Assessment was performed by the clinician to evaluate the lesion/pimple redness (erythema) on a 4 point scale where 0 meant none, 1 meant mild, 2 meant moderate and 3 meant severe. The size and the height (elevation, swelling) of the lesion/pimple was also evaluated on a 4-point scale (0-3) where 0 meant non/flat and 3 meant severe/prominent. Half points were used when necessary for evaluation of erythema, size and swelling. Mean values for clinical grading at hours 4, 8 and 24 after product application were statistically compared to mean baseline values for significant differences using a paired t-test at the p<0.05 significance level. The results are shown in Table 2.

TABLE 1

| Ingredient | Amount (wt %) |
|---|---|
| Purified Water | 1.12888 |
| Hamamelis Virginiana (Witch | 37.6300 |
| Hazel) Bark Leaf/Twig Extract | |
| SDAG-3 Alcohol 95% | 37.300 |
| Polyquaternium 37 | 4.000 |
| Glycerin | 3.000 |
| Glycolic Acid (70%) | 4.200 |
| Butylene Glycol | 2.000 |
| Salicylic Acid | 2.000 |
| Cyclopentasiloxane (and) | 1.670 |
| Cyclopentasiloxane | |
| C12-15 Alkyl Lactate | 1.100 |
| Cocamidopropyl PG-Dimonium | 1.000 |
| Chloride Phosphate | |
| Isodecyl Laurate | 1.000 |
| Sodium Hydroxide 50% | 1.800 |
| Bisabolol | 0.600 |
| Cetyl Lactate | 0.550 |
| Polysorbate 20 | 0.500 |
| Benzalkonium Chloride (50%) | 0.200 |
| Fragrance, | 0.200 |
| Portuluca Oleracea Extract (and) | 0.100 |
| Butylene Glycol (and) Water | |
| Sodium Benzotriazolyl | 0.020 |
| Butylphenol Sulfonate | |
| Yellow 6 | 0.00098 |
| Red 40 | 0.00014 |
| Total | 100.00% |

TABLE 2

| Speed of resolution | 4 hours | 8 hours | 24 hours |
|---|---|---|---|
| Erythema | −7.60% | −11.50% | −29.30% |
| Lesion Size | −6.00% | −14.80% | −36.80% |
| Elevation (Swelling) | −15.90% | −29.30% | −49.70% |

The data in Table 2 shows that active pimples begin resolution using the inventive composition within about 4 hours after application. The percentages shown in Table 2 are the average percentage change in condition (redness, size and swelling) from baseline taken at each time period. Negative numbers indicate improvement from baseline. Some erythema of the surrounding skin was present, which is typical of acne treatment products.

### Example 2

An in vivo clinical study of the inventive composition according to Table 1 above was conducted on 25 human volunteers. The study was double blind and randomized. The subjects suffered from acne. An acne lesion on the face of each subject was evaluated by a clinician and the subject at baseline. The subjects then applied the inventive composition one to two times per day to the lesion. The treatment site was evaluated at day 2 and weeks 1, 2, 4 and 8 by the subjects (self assessment) and the clinician.

TABLE 3

| | Week 4 |
|---|---|
| | Percent of Subjects |
| Mark/Scar characteristic | Improved |
| Color Intensity (N = 18) | 75.00% |
| Size (N = 18) | 61.11% |
| Scar Severity (N = 8) | 90.00% |

The percent of subjects showing color intensity fading, size reduction and scar healing after 4 weeks is shown in Table 3.75% of the subjects using the inventive composition showed color intensity fading of the acne marks or scar at week 4. Table 3 also indicates that 61.11% of the subjects showed reduction in the size of the scar and 90% of the subjects showed improved healing of the scar after using the inventive composition.

Table 4 shows the data for clinical evaluation of the overall skin tone/evenness percentage change using the inventive composition on the face at day 2, week 1, week 2, week 4 and week 8. The skin tone/evenness was graded on the face using a 4-point scale (0-3) where 0 meant even tone and 3 meant severe/unevenness. The values shown in table 4 were generated by calculating the average score of the subjects' overall skin tone/evenness recorded at baseline and the average score of the subjects' overall skin tone/evenness recorded at each time frame (day 2, week 1, week 2, week 4 and week 8) and then compared to calculate the percentage of change. The negative numbers indicate improvement from baseline, and were significant at the p<0.05 significance level.

TABLE 4

| | Day 2 | Week 1 | Week 2 | Week 4 | Week 8 |
|---|---|---|---|---|---|
| % Δ | (N = 23) | (N = 23) | (N = 21) | (N = 20) | (N = 20) |
| Overall | −3.30% | −11.60% | −21.40% | −16.90% | −21.80% |
| Skin Tone | | | | | |
| Uneveness | | | | | |

Table 5 shows data for the self-assessment of overall skin condition at different treatment time points. The values shown are percentages of subjects who strongly agree or somewhat agree to the statements.

TABLE 5

| Top Two Boxes | Day 2 | Week 1 | Week 2 | Week 4 | Week 8 |
|---|---|---|---|---|---|
| My post acne marks | 47.8% | 69.5% | 62.0% | 75.0% | 66.6% |
| look reduced/become | | | | | |
| less visible | | | | | |
| My acne scars look | 22.0% | 48.0% | 48.0% | 75.0% | 57.0% |
| reduced/become | | | | | |
| less visible | | | | | |
| The acne areas on | 48.0% | 86.9% | 62.0% | 70.0% | 67.0% |
| my face look less | | | | | |
| red | | | | | |
| My skin tone looks | 13.0% | 60.8% | 38.0% | 60.0% | 48.0% |
| more even | | | | | |
| My complexion | 30.0% | 65.2% | 38.0% | 60.0% | 52.0% |
| looks clearer | | | | | |
| My skin feels/ | 26.0% | 65.2% | 47.0% | 65.0% | 52.0% |
| looks smoother | | | | | |
| My skin looks less | 26.0% | 60.8% | 38.0% | 55.0% | 52.0% |
| shiny and feels | | | | | |
| less oily | | | | | |
| My skin feels/ | 9.0% | 56.5% | 48.0% | 55.0% | 61.9% |
| looks improved | | | | | |

Table 5.1 shows data for self-assessment of a target pimple after using the inventive composition. The numbers indicate the percentages of subjects who strongly agreed or somewhat agreed to the statements.

TABLE 5.1

| Top Two | Day 2 | Week 1 | Week 2 | Week 4 | Week 8 |
|---|---|---|---|---|---|
| Boxes | (N = 23) | (N = 23) | (N = 21) | (N = 20) | (N = 21) |
| My target | 52.1% | 86.9% | 90.4% | 90.0% | 90.4% |
| pimples look | | | | | |
| smaller | | | | | |
| My target | 43.4% | 86.9% | 80.9% | 85.0% | 80.9% |
| pimples look | | | | | |
| less red | | | | | |
| My target | 43.4% | 95.6% | 76.1% | 80.0% | 85.7% |
| pimples look | | | | | |
| less swollen | | | | | |
| My target | 43.4% | 95.6% | 90.4% | 85.0% | 85.7% |
| pimples look | | | | | |
| less severe | | | | | |
| My target | 47.8% | 86.9% | 71.4% | 75.0% | 76.1% |
| pimples look | | | | | |
| less visible/ | | | | | |
| noticeable | | | | | |
| My pimples | 43.4% | 60.8% | 57.1% | 70.0% | 61.9% |
| are resolving | | | | | |
| faster than | | | | | |
| before | | | | | |
| treatment | | | | | |

Table 5.2 shows the data for self-assessment of treatment benefits of using the inventive composition. The subjects assessed the treatment benefit based on a 10 point scale ranging from: not at all noticeable (0) to very noticeable (9). The results indicate the average percentage change of self assessed severity from baseline.

TABLE 5.2

| Questionnaire - Change from | | Day 2 | Week 1 | Week 2 | Week 4 | Week 8 |
|---|---|---|---|---|---|---|
| Baseline | | (N = 23) | (N = 23) | (N = 21) | (N = 20) | (N = 21) |
| Whole | Looking at the | −8.6% | −16.6% | −14.5% | −21.0% | −13.3% |
| Face | pimples currently on | | | | | |
| | your face please | | | | | |
| | indicate how | | | | | |
| | noticeable your | | | | | |
| | pimples look to you | | | | | |
| | right now? | | | | | |
| | Looking at the | −7.3% | −11.6% | −13.3% | −21.1% | −17.3% |
| | pimples on your face | | | | | |
| | indicate on average | | | | | |
| | how red they look to | | | | | |
| | you? | | | | | |
| | Looking at your face | −7.1% | −23.7% | −22.0% | −24.7% | −15.7% |
| | please indicate how | | | | | |
| | oily and shiny you | | | | | |
| | feel your face is? | | | | | |
| Target | How would you | −16.4% | −37.9% | −38.7% | −42.4% | −43.6% |
| Pimples | describe the severity | | | | | |
| | of the target | | | | | |
| | pimples? | | | | | |
| | Please indicate how | −19.5% | −33.5% | −42.9% | −45.6% | −50.0% |
| | noticeable you feel | | | | | |
| | those pimples are to | | | | | |
| | you. | | | | | |
| | Indicate on average | −16.0% | −34.0% | −41.6% | −48.6% | −47.9% |
| | how red you feel are | | | | | |
| | those target pimples. | | | | | |
| | Indicate on average | −10.6% | −31.4% | −39.4% | −45.1% | −51.7% |
| | how big you feel the | | | | | |
| | target pimples look. | | | | | |
| | Indicate on average | −10.0% | −32.7% | −35.3% | −38.2% | −46.5% |
| | how swollen you feel | | | | | |
| | the target pimples | | | | | |
| | look. | | | | | |

Table 5.3 shows the data for the level of irritation evaluated by clinical grading using a 4 point scale, where 0 meant no irritation and 3 meant severe irritation.

TABLE 5.3

| | Baseline | Day 2 | Week 1 | Week 2 | Week 4 | Week 8 |
|---|---|---|---|---|---|---|
| Erythema | 0.52 | 0.48 | 0.52 | 0.45 | 0.50 | 0.45 |
| Edema | 0.00 | 0.00 | 0.00 | 0.00 | 0.00 | 0.00 |
| Scaling | 0.00 | 0.07 | 0.04 | 0.00 | 0.00 | 0.00 |
| Stinging | 0.00 | 0.00 | 0.00 | 0.02 | 0.08 | 0.00 |
| Burning | 0.00 | 0.00 | 0.00 | 0.00 | 0.00 | 0.02 |
| Itching | 0.04 | 0.00 | 0.00 | 0.00 | 0.00 | 0.02 |

While this specification describes the principles of the present invention, it is to be understood that other aspects, advantages and modifications within the scope of the invention will be apparent to those skilled in the art.

What is claimed is:

1. A composition for treating acne comprising an alpha-hydroxy acid, an anti-acne agent, an anti-microbial agent and a lactate, wherein said composition is capable of reducing an appearance of acne within about one week.

2. The composition according to claim 1, wherein said alpha-hydroxy acid is selected from glycolic acid, lactic acid, malic acid, citric acid, tartaric acid and combinations thereof.

3. The composition according to claim 1, wherein said alpha-hydroxy acid is present in an amount of about 0.5% to about 5.0% by weight of the composition.

4. The composition according to claim 3, wherein said alpha-hydroxy acid comprises glycolic acid.

5. The composition according to claim 4, wherein said glycolic acid is present in an amount of about 1.5% to about 4.0% by weight of the composition.

6. The composition according to claim 4, wherein said glycolic acid is present in an amount of about 2.5% to about 3.0% by weight of the composition.

7. The composition according to claim 1, wherein said anti-acne agent is selected from the group consisting of salicylic acid, benzoyl peroxide, sulphur, retinoic acid, azelaic acid, clindamycin, adapalene, erythromycin, sodium sulfacetamide, dapsone and combinations thereof.

8. The composition according to claim 1, wherein said anti-acne agent is present in an amount of about 0.1% to about 10% by weight of the composition.

9. The composition according to claim 1, wherein said anti-microbial agent is selected from the group consisting of benzalkonium chloride, benzethonium chloride, cetylpyridinium chloride, 3-iodo-2-propynyl-N-butylcarbamate, hexetidine (5-amino-1,3-bis-(2-ethylhexyl)-5-methyl-hexahydropyrimidine), Quaternium 15 triclosan, chlorhexidine digluconate, and combinations thereof.

10. The composition according to claim 1, wherein said anti-microbial agent is present in an amount of about 0.001% to about 10% by weight of the composition.

11. The composition according to claim 1, wherein said lactate is selected from the group consisting of C₁₂-C₁₆ alkyl lactates and combinations thereof.

12. The composition according to claim 1, wherein said lactate is present in an amount of about 0.1% to about 50% by weight of the composition.

13. The composition according to claim 1 wherein said composition further comprises a phospholipid.

14. The composition according to claim 13 wherein said phospholipid is selected from the group consisting of sodium coco PG-dimonium chloride phosphate, cocamidopropyl PG-dimonium chloride phosphate, myristamidopropyl PG-dimonium chloride phosphate and combinations thereof.

15. A method of reducing the appearance of an acne scar comprising applying to an area of skin in need of such treatment a composition comprising an alpha-hydroxy acid, an anti-acne agent, an antimicrobial agent, and a lactate.

16. A method according to claim 15, wherein the appearance of said acne scar is reduced within about a week.

17. A method of reducing the size of an acne lesion comprising applying to an area of skin in need of such treatment a composition comprising an alpha-hydroxy acid, an anti-acne agent, an antimicrobial agent, and a lactate.

18. A method according to claim 17, wherein said acne lesion is reduced in size by about 6.0% within about 4 hours.
